(19) United States
(12) Patent Application Publication
(10) Pub. No.: US 2003/0114415 A1
(43) Pub. Date: Jun. 19, 2003

(54) Compositions and methods for treating and preventing memory impairment using citicoline

(76) Inventors: Richard J. Wurtman, Boston, MA (US); Lisa Teather, Watertown, MA (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/073,272

(22) Filed: Feb. 13, 2002

Related U.S. Application Data
(60) Provisional application No. 60/339,445, filed on Dec. 14, 2001.

(51) Int. Cl. A61K 31/7072; A61K 31/202; A61K 31/513

(52) U.S. Cl. 514/51; 514/560

(57) ABSTRACT

This invention relates to compositions and methods for preventing and treating cognitive dysfunction or memory impairment. The compositions include an effective amount of citicoline, or pharmaceutically-acceptable salts thereof, and one or more of the compounds selected from the group consisting of linoleic acid and linolenic acid. Other compositions of this invention include an effective amount of citicoline, or pharmaceutically-acceptable salt thereof, wherein said citicoline is metabolized to form at least one of cytidine, uridine, and choline. Still other compositions of this invention include effective amounts of choline, cytidine, and/or uridine, or their pharmaceutically-acceptable salts. This invention also encompasses methods for preparing these compositions.

This application claims priority from U.S. Serial No. 60/339,445, filed Dec. 14, 2001.

### 1. FIELD OF THE INVENTION

This invention relates to compositions and methods for preventing and/or reducing memory impairment, particularly memory impairment caused by Mild Cognitive Impairment (MCI) or more severe dementias such as Alzheimer's Disease (AD), or other disorders including cerebrovascular disease. Administration of the compounds of this invention may enhance acetylcholine and membrane phosphatidyl choline synthesis, thereby increasing the amount of brain neurotransmitters available, and enhancing the growth of brain cells. More particularly, the invention relates to the use of compositions containing citicoline (cytidine-5′-diphosphocholine or CDP-choline) or its metabolites choline, cytidine, and/or uridine, and optionally one or more fatty acids, in a novel treatment regimen to prevent or reduce memory impairment and improve cognitive function and quality of life for patients suffering from these conditions.

### 2. BACKGROUND OF THE INVENTION

Memory impairment can be caused by a variety of disorders, and can range from mild to severe. The memory loss may be temporary or permanent, and may be the result of brain damage (either due to disease or trauma), depression, use of certain medications, emotional trauma, physiological changes in the brain, and reduction in the ability of the central nervous system to process information. Major theories held by neurologists believe that gradual deterioration of the brain causes memory loss. As people age, some neurons fail to function properly, or deteriorate to a non-functioning point, causing some problems with memory.

Age-Associated Memory Impairment (AAMI) refers to a decline in memory due to aging, characterized by temporary memory lapses in otherwise healthy individuals. Memory is commonly divided into two parts, short term and long term. Long term memory is affected less by aging than short term memory. Individuals with AAMI exhibit lapses in short term memory that are within the limits of what is considered “normal” for their age group. Such changes in memory in later life are often due to a combination of influences, including physiological changes in the brain, slowdowns in central nervous system processing abilities, certain diseases, and the use of certain medications (e.g., Aldomet (methyldopa), Ascendin (amoxapine), Dalmane (flurazepam), Elavil (amitryptyline), Equanil (meprobamate), Haldol (haloperidol), Inderal (propranolol), Mellaril (thioridazine), Miltown (meprobamate), Pamelor (nortryptyline), Pepcid (famotidine), Seraz (oxazepam), Symmetrel (amantadine), Tagamet (cimetidine), Valium (diazepam), and Xantac (ranitidine)). Other reasons may be reduced motivation to remember, or simple disuse. It may be possible for individuals with AAMI to improve their memories by engaging in mental exercise, improving their diet, and incorporating exercise into their lifestyles.

Memory decline that is more severe or consistent than AAMI may be classified as Mild Cognitive Impairment (MCI), and, in a minority of patients, indicates the early stages of a condition such as dementia. The memory lapses of MCI are more severe than age-related forgetfulness. In memory tests, people with MCI retain less information than most people their age. This memory impairment is also persistent, interfering with normal daily routines. According to studies, about 12 percent of people age 65 or older who were previously diagnosed with MCI are diagnosed with AD every year, making MCI one of the most important risk factors for AD.

The decline from MCI to Alzheimer's disease (AD) involves significant degradation of cognitive function, both in terms of the kinds of problems encountered and their severity. Patients with AD show abnormal memory impairment for their age group, as well as additional impairments in other mental skills. Loss of cholinergic neurons within the nucleus basalis has been correlated with cognitive impairment and disease severity through studies conducted using postmortem tissue from AD patients. AD is considered a form of dementia, which has symptoms including difficulties with language, learning, thinking and reasoning, as well as memory loss, and may also result in changes in mood and personality, eventually becoming severe enough to interfere with a person's work, everyday activities and social life. Other irreversible dementias include Parkinson's disease, Huntington's disease, Creutzfeldt-Jakob disease and multi-infarct dementia. Some reversible causes of dementia include nutritional and vitamin deficiencies, drug intoxication, thyroid and blood chemistry imbalances, and tumors.

Cerebrovascular disease is also potentially devastating in terms of its effects on memory, and it is more common than AD. More than 700,000 Americans suffer a major cerebrovascular event—usually a stroke—each year. Stroke is the third leading cause of death in the United States and the number one cause of disability with more than 3,000,000 currently living with permanent brain damage, including memory loss, caused by such an event. The term cerebrovascular disease covers acute stroke and other diseases that may lead to stroke, like arteriovenous malformations (AVM), aneurysms, craniofacial venous malformations, brain tumors, spinal tumors, stenotic and thromboembolic occlusive diseases, and moyamoya. All of these cerebrovascular diseases have the potential to cause memory impairments, as well as other cognitive dysfunctions.

Various pharmaceutical therapies have been developed to combat memory loss, particularly memory loss associated with AD. These include:

Tacrine, or Cognex, which was the first drug approved in the US for the treatment of AD. It slows progression of AD by increasing levels of the neurotransmitter acetylcholine. It needs to be taken four times a day and blood tests for liver function need to be monitored. Up to six out of ten people are unable to reach the maximum dosage due to side effects.

Donepezil, or Aricept, is a widely known drug that was approved by the Food and Drug Administration (FDA) over four years ago for the treatment of memory loss related to AD. The drug raises the level of the chemical acetylcholine in the brain. By doing so, it slows decline of the disease. Recently, it has been used in other kinds of dementias as well as reversible memory loss. There is growing literature about its use in various conditions including head trauma, mild cognitive impairment and other conditions. Side effects include gastrointestinal discomfort.

Rivastigmine, or Exelon, was approved by the FDA to treat AD. It also increases levels of acetylcholine in the brain. It is given twice a day and side effects include gastrointestinal discomfort.

Memantine, or Akatinol, is an NMDA receptor agent that promotes nerve cell viability. It has been used in European countries including Germany to treat memory loss, but has not been approved for use in the US. Limited trials are ongoing at this time in the US.

Galantamine, or Reminyl, is an agent that raises brain levels of acetylcholine. Some preliminary data suggests that is more effective than earlier cholinergic agents. It is currently in limited US clinical trials and is not approved by the FDA. It has been in widespread use in Europe.

Neotropin may promote the growth of nerve cells and maintain nerve cell viability. It is currently being used in trials abroad, but not in the US.

Nootropics, the first class of agents used for treatment of memory loss, have not been shown to be consistently effective and are not used routinely in the US.

Alpha-tocopherol, or vitamin E, in doses of 2000 IU has been shown to slow progression of Alzheimer's disease. The drug is believed to work as a free radical scavenger, and promote nerve cell viability.

Selegeline, or Eldepryl, is an agent that both raises the levels of certain neurochemicals and promotes nerve cell viability, and has been used in the US for the treatment of Parkinson's disease. It has been shown to be effective for the treatment of Alzheimer's.

Non-steroidal anti-inflammatory agents, or NSAIDS, include drugs such as ibuprofen (e.g., Motrin, Advil), as well as the newer cyclo-oxygenase 2 inhibitors (e.g., Celebrex and Vioxx). There is preliminary evidence to suggest that they may be helpful in treating some types of memory loss. Limited clinical trials are ongoing in the US.

Gingko Biloba, a free radical scavenger and possible brain activator, is commonly prescribed for the treatment of dementia in Europe. Preparations of the drug in the US vary, and the right dose of the right preparation may slow progression of some types of memory loss.

Estrogen therapy may help to prevent Alzheimer's disease in women.

B-secretase inhibitors are the newest class of drugs being developed for treating memory loss. These drugs stop formation of amyloid plaques, and may halt the progression of illnesses like Alzheimer's. These drugs are currently involved in trials abroad.

Certain classes of the B vitamins (particularly B₆ and B₁₂) are felt to be neuroprotective, and are being used in clinical trials for treating memory loss.

Calcium channel blockers, a class of drugs used to treat illnesses like hypertension and migraine, have been used to treat memory loss.

Citicoline monosodium is an exogenous form of cytidine-5′-diphosphocholine (CDP-choline). Endogenous CDP-Choline is a key intermediate in the biosynthesis of membrane phosphatidyl choline, which is of primary importance for the dynamic regulation of cellular integrity. The role of phospholipids in the maintenance of neuronal function is critically important in conditions such as MCI, AD, and various cerebrovascular diseases, where the breakdown of these membranes is thought to contribute to memory impairment. In addition to its role in neuron membrane structural function, phosphatidyl choline is thought to play a major role in lipid turnover (utilization of fatty acids) and communication signaling. It also acts as a neuroprotector. Citicoline donates the components choline, cytidine, and uridine (precursors to the synthesis of phosphatidylcholine), required to form, repair, and even restore function to nerve cell membranes. Cytidine and uridine, acting through cytidyl triphosphate (CTP), are also involved in the synthesis of other phospholipids. In addition, choline promotes the synthesis of acetylcholine, a neurotransmitter intimately associated with cognition. As an information-transmitting molecule, acetylcholine is necessary for proper memory function and is especially important for aging brains. In the brain, in addition to promoting phospholipid synthesis, citicoline also inhibits phospholipid degradation. Citicoline's mechanism of action is thought to entail cerebrovascular (blood circulation of the brain) regulation and neuroimmune (immune function of the nervous system) actions in the brain.

Citicoline has been extensively studied in clinical trials. Results of these trials indicated an improvement in a variety of clinical symptoms, including headache, vertigo, motor coordination and insomnia. These trials also showed improvements in motor function and reduction in stroke sequelae. However, such trials were limited to the use of citicoline during the rehabilitation stage of patients who may have suffered a stroke, and, thus, such treatments occurred well after the putative ischemic event. Nevertheless, such trials are informative for purposes of this invention because they show that stroke and head trauma patients tolerated citicoline well at dose ranges of 250 mg/day to 1000 mg/day for several weeks.

Although many pharmaceutical products have been developed in order to combat conditions that cause memory impairment, and particularly to slow or halt the progression of AD, none are highly effective in this regard. Further, none have been demonstrated to be effective in preventing the onset of symptoms of memory impairment. Accordingly, a need exists in the art for a composition and method for preventing or treating memory impairment.

### 3. SUMMARY OF THE INVENTION

The invention relates to compositions and methods for preventing and treating memory impairment, particularly memory impairment caused by any of a number of disorders, such as stroke, brain injury, mild cognitive impairment (MCI), Alzheimer's Disease (AD), cerebrovascular disease, and other disorders which cause cognitive disturbances. The compositions according to this invention include citicoline, or its metabolism products, choline, cytidine, and/or uridine, and optionally one or more fatty acids such as linoleic acid and linolenic acid, and their active metabolites, e.g., arachidonic acid and docosohexenoic acid and any other essential fatty acids that are metabolized to form diacylglycerol (DAG). The methods include administering an effective amount of citicoline or a pharmaceutically-acceptable salt thereof, optionally in conjunction with these fatty acids, or administering any combination of choline, cytidine, and uridine, or the pharmaceutically-acceptable salts thereof, optionally in combination with these fatty acids.

The present invention also relates to the use of citicoline for the preparation of a pharmaceutical medicament for the prevention and treatment of memory impairment, comprising admixing an effective amount of citicoline, optionally with one or more fatty acids and a pharmaceutically acceptable carrier or as components of a food, or comprising admixing effective amounts of choline, cytidine, and/or uridine, optionally with one or more fatty acids and a pharmaceutically-acceptable carrier.

In the method of preventing and treating memory impairment, administration of an effective amount of citicoline preferably takes place over a specified period, typically over at least several weeks (e.g., 3-8 weeks, preferably at least about 6 weeks), and most preferably for an indefinite period. The dosage regimen can vary within certain limits. Typically, about 40-4000 mg of citicoline can be administered one or more times per day for the duration of the treatment period. The amount administered to a particular patient may vary based on several factors known to those of skill in the art, such as age, weight, severity of cognitive dysfunction, etc. The preferred dosage includes about 10-1000 mg of citicoline up to four (4) times a day, preferably about 2000 mg in a single dose or in divided doses. If choline, cytidine, and uridine are used, they are preferably provided in amounts commensurate with the amount of these compounds that are released when citicoline is metabolized. If fatty acids, such as linoleic acid and/or linolenic acid, are included, they are preferably provided in amounts thought to be sufficient to provide a beneficial amount of brain DAG.

Citicoline may be expected to have a number of advantages over other agents being developed for the prevention and treatment of memory impairment. Being an endogenous compound, citicoline is inherently safe. Citicoline has a very low toxicity and an extremely broad therapeutic index. The same applies to combinations of choline, cytidine, and uridine, as these are the natural by-products of the metabolism of citicoline in mammals. Fatty acids such as linoleic acid and linolenic acid are also low in toxicity, and are natural components of the diet of most animals.

The potential multimodal action of citicoline also may prove advantageous. Although the relative contribution of each potential mode of action to the treatment of cognitive dysfunction is not known, citicoline and its hydrolysis products—cytidine, uridine and choline—are believed to play important roles in the generation of phospholipids involved in membrane formation and repair, as well as synthesis of acetylcholine, which is a brain neurotransmitter. These compounds also are believed to contribute to critical metabolic functions, such as the formation of nucleic acids and proteins. See, Ulus, I. H. et al. Brain Research (1989) 484:217-227. Thus, under conditions where memory impairment occurs, citicoline may function to (1) stabilize membranes by providing substrate for membrane maintenance; (2) repair damaged membranes by supplying important substrates for membrane formation; and (3) restore neuronal function by supplying substrate for the formation of acetylcholine. Moreover, unlike other proposed therapeutic agents, citicoline has the potential not only to prevent deterioration in cognitive ability, but also to contribute to the treatment of damaged areas of the brain, e.g., after stroke brain injury, thereby improving or restoring cognitive function.

It is, therefore, one object of the invention to provide a composition for preventing or treating memory impairment, comprising an effective amount of citicoline, or pharmaceutically-acceptable salt thereof, wherein said citicoline is metabolized to form at least one of cytidine, uridine, and choline.

Yet another object of the invention is to provide a composition for treating or preventing cognitive dysfunction, comprising an effective amount of citicoline, or pharmaceutically-acceptable salt thereof, and one or more of the compounds selected from the group consisting of linoleic acid and linolenic acid or their active metabolites.

Still another object of this invention is to provide a composition for preventing or treating memory impairment, comprising at least one of an effective amount of choline or a pharmaceutically-acceptable salt thereof, an effective amount of cytidine or a pharmaceutically-acceptable salt thereof, and an effective amount of uridine or a pharmaceutically-acceptable salt thereof.

A further object of the invention is to provide a method for preparing a composition for use in treating or preventing memory impairment, comprising the steps of providing an effective amount of citicoline, or a pharmaceutically-acceptable salt thereof, providing an effective amount of one or more second compounds selected from the group consisting of linoleic acid and linolenic acid, and combining said citicoline with said one or more second compounds to form a pharmaceutically-acceptable preparation.

Another aspect of this invention is to provide a method for preparing a composition for use in treating of preventing memory impairment, comprising the steps of providing at least one of an effective amount of choline or a pharmaceutically-acceptable salt thereof, providing an effective amount of cytidine or a pharmaceutically-acceptable salt thereof, providing an effective amount of uridine or a pharmaceutically-acceptable salt thereof.

A further aspect of this invention is to provide a method of preventing or treating cognitive disorders, comprising the steps of administering at least one of an effective amount of choline or a pharmaceutically-acceptable salt thereof, administering an effective amount of cytidine or a pharmaceutically-acceptable salt thereof, administering an effective amount of uridine or a pharmaceutically-acceptable salt thereof; and continuing to administer said citicoline for a period of at least about six weeks.

An additional object of this invention is to provide a method of preventing or treating memory impairment, comprising the steps of administering an effective amount of citicoline, or a pharmaceutically-acceptable salt thereof, combined with one or more of the compounds selected from the group consisting of linoleic acid and linolenic acid, and continuing to administer said citicoline for a period of at least about six weeks.

These and other objects and aspects of the invention will be apparent to those of ordinary skill in view of the discussion above and the additional detailed description provided below relating to preferred embodiments of the invention.

### 4. BRIEF DESCRIPTION OF THE DRAWINGS

FIG. 1 depicts a graph comparing the mean times taken by four groups of rats to acquire a hidden target. Rats were grouped according to whether they were reared in restricted or enriched conditions, and whether they received a diet supplemented with CDP-choline and unsaturated acids or a standard diet.

FIG. 2 depicts a graph comparing the mean times taken by the same four groups of rats to acquire a visible target.

FIG. 3 depicts a bar graph comparing the mean times taken by the same four groups of rats to acquire a visual discrimination task.

FIG. 4 depicts bar graphs comparing the mean correct responses given by the same four groups of rats when presented with distracting stimuli during a learned stimulus-response task.

FIG. 5 depicts a bar graph comparing the mean correct responses given by four groups of rats after originally learning relevant information while being presented with additional irrelevant information.

FIG. 6 depicts a bar graph comparing the amounts of phosphatidylcholine produced by CHP134 cells that were administered a choline-containing medium that also contains one of uridine, diacylglycerol, or a combination of uridine and diacylglercerol, as compared to control cells receiving only choline-containing medium.

FIG. 7 depicts a bar graph comparing the amounts of CDP-choline remaining in CHP134 cells that were administered a choline-containing medium that also contains one of uridine, diacylglycerol, or a combination of uridine and diacylglycerol, as compared to control cells receiving only choline-containing medium.

### 5. DETAILED DESCRIPTION OF THE PREFERRED EMBODIMENTS

This invention comprises compositions and methods for preventing and/or reducing memory impairment caused by MCI or AD, or by any of a number of other conditions, including those caused by cerebrovascular disease. The compounds of this invention include citicoline, and/or any combination of choline, cytidine, and uridine, which are believed to increase production of membrane phosphatidyl choline and acetylcholine, thereby enhancing the growth of brain cells and increasing the availability of neurotransmitters. The compounds of this invention may also optionally include one or more fatty acids, such as linoleic acid or linolenic acid, or any other fatty acid that is metabolized to form diacylglycerol (DAG).

Without being limited by theory, it is believed that citicoline and its metabolites have at least a multiple mechanism of action: aiding in the repair of damaged neuronal tissues, enhancing the growth of axons and synapses, and aiding in the synthesis of brain neurotransmitter acetylcholine. Administration of citicoline is also believed to repair tissue damage by preventing the accumulation of potentially toxic free fatty acids which can be oxidized to generate free radicals, and the activation of enzymes that liberate these fatty acids. Upon oral or parenteral administration, CDP-choline releases its two principle components, cytidine and choline.

In humans, most of the cytidine is converted to uridine, which is phosphorylated to form uridine triphosphate (UTP), that is then converted to cytidine triphosphate (CTP). In addition, the choline is converted to its intermediate phosphocholine by the enzyme choline kinase. The CTP and phosphocholine together form CDP-choline in the brain, and the presence of DAG allows formation of phosphatidylcholine, a major neuronal membrane constituent. To promote availability of DAG, it is beneficial to administer a fatty acid such as linoleic acid or linolenic acid in conjunction with the citicoline, as these fatty acids are metabolized in part to form brain DAG. It is believed that DAG enhances the activity of choline phosphotransferase, although the invention is not bound to any particular theory. See Araki & Wurtman, Proc. Nat'l. Acad. Sci. U.S.A. 97: 11,946-11,950.

When administered orally, CDP-choline is absorbed almost completely, and its bioavailability is approximately the same as when administered intravenously. Once absorbed, it is metabolized to cytidine, uridine and choline which are dispersed widely throughout the organism, crossing the blood-brain barrier and reaching the central nervous system (CNS), where they are incorporated into the phospholipid fraction of the membrane and microsomes. CDP-choline activates the biosynthesis of structural phospholipids in the neuronal membranes, concurrently increasing cerebral metabolism and acting on the levels of various neurotransmitters. Due to these pharmacological activities, CDP-choline has a neuroprotective effect in situations of hypoxia and ischemia, and results in improved learning and memory performance in animal models of brain aging.

It is further postulated that to normalize brain function, nerve cells damaged by disorders affecting cognitive ability must manufacture new membrane elements, and the amount of neurotransmitters present in the brain must be increased. As described below, in preclinical animal models of cognitive impairment, administration of CDP-choline as a dietary supplement is shown to significantly improve cognitive function. It should be noted that the CDP-choline was also administered in conjunction with both linoleic acid and linolenic acid.

This invention is directed to a new and important use of citicoline and its metabolites: the treatment and prevention of memory impairment caused by any of a number of underlying conditions. Although stabilization of membranes and increased production of acetylcholine are believed to be of benefit in treating cognitive dysfunction, it has not been definitively demonstrated that production of additional neurotransmitters and stabilization of membranes will improve cognitive function. The inventors have unexpectedly found that administration of citicoline significantly improves cognitive function, presumably by altering phosphatidylcholine synthesis and membrane formation and stimulating acetylcholine production, although this invention is not limited to any particular mechanism of action.

The citicoline-based compositions of this invention are generally preferred to be administered orally as a pharmaceutically-acceptable salt thereof. The preferred salt is the monosodium salt of citicoline, as this form is readily available in pharmaceutically-acceptable purity, although use of other pharmaceutically-acceptable citicoline salts is also envisioned. Further, where any combination of choline, cytidine, and uridine is used, it is also preferably administered orally as a pharmaceutically-acceptable salt or in a food.

Treatment under the invention is preferably begun prior to the onset of cognitive impairment symptoms, such as memory loss, e.g., in people who have had a stroke or brain injury, or shortly after such symptoms are first exhibited. However, the compositions and methods of this invention may also be beneficially administered to patients exhibiting advanced cognitive impairment in order to improve their cognitive function. In a specific embodiment of the invention, treatment is continued for at least up to about several weeks, preferably at least up to about several months, and most preferably for an indefinite period after the start of treatment.

Hence, according to one embodiment of the invention, a method is disclosed of treating memory impairment, and preventing future impairment, in a patient who has a disorder that causes cognitive dysfunction, comprising administering an effective amount of citicoline or a pharmaceutically acceptable salt thereof over a period of at least several weeks. Preferably, a fatty acid such as linoleic acid or linolenic acid is administered in conjunction with the citicoline. Preferably, the doses are administered at least once per day, and the treatment begins prior to the onset of symptoms of cognitive impairment. The preferred dose of about 40 to about 4000 mg of citicoline or its pharmaceutically acceptable salt may be administered one or more times daily. Alternatively, the method of this invention may comprise administering an effective amount of a combination of any of choline, cytidine, and uridine, or pharmaceutically-acceptable salts thereof, optionally in conjunction with administration of one or more fatty acids, over a period of at least several weeks.

The method of this invention finds its most advantageous use in human patients who have a disorder that results in memory impairment and other cognitive dysfunctions, including patients with MCI and AD. It cannot be stressed enough, however, that the citicoline, or the choline, cytidine, and uridine combinations, should be administered as soon as possible after symptoms of impairment begin to be exhibited. Further, citicoline, or the choline, cytidine, uridine combinations, may be administered as a preventative measure to patients at risk for developing disorders that cause cognitive impairment. It is beneficial to co-administer these compositions with essential fatty acids that are metabolized to form DAG, such as linoleic acid or linolenic acid.

A variety of dosage ranges are suitable. The citicoline dosage under the invention may be from about 10 mg to about 1000 mg from one to about 4 times per day. For example, when a single daily dose is desired, citicoline is administered in from about 40 to about 4000 mg per day, preferably from about 500 to about 2000 mg per day. In one embodiment of the invention, the dose is 1000 mg per day. When choline and cytidine and/or uridine are used, the dose is preferably sufficient to provide these compounds in amounts that are commensurate with the amounts of choline, cytidine and/or uridine released when citicoline is metabolized.

For the composition of this invention, the amount of citicoline, or a pharmacologically-acceptable salt thereof required to achieve a therapeutic effect will vary with the route of administration and the particular disorder or disease to be treated. A suitable systemic dose of the active ingredient for a mammal suffering from, or likely to suffer from, any of the conditions described herein, is in the range of 40 mg to 4000 mg per day, with a preferred dose of 2000 mg per day. A dose of 1000 mg citicoline per day in a human will produce a plasma choline concentration of 1.5 ng/ml, the same as that produced by the administration of 500 mg/kg/day citicoline to the rat. It has been shown, however, that 500 mg per day conveys most of the benefits of citicoline treatment while minimizing any negative side effects, including dizziness, which may be experienced by some patients.

While it is possible for citicoline, or a combination of choline and cytidine and/or uridine to be administered alone, and in combination with fatty acids such as linoleic acid and linolenic acid, it may be preferable to present the active ingredients of the compositions of this invention as a formulation. Formulations of the active ingredients suitable for oral administration may be in the form of discrete units, such as capsules, cachets, tablets, or lozenges; in the form of a powder or granules for reconstitution; in the form of a solution or a suspension in an aqueous liquid or nonaqueous liquid; or, in the form of an oil-in-water emulsion or a water-in-oil emulsion or in a food base, e.g., pasta. The active ingredient also may be in the form of a bolus, electuary, or paste. Formulations of the active ingredient suitable for parenteral administration may comprise a sterile, aqueous preparation of the active ingredient. The formulations may be presented in unit dosage form and may be prepared by any of the methods well-known in the art of pharmacology.

In addition to containing the standard and well known pharmaceutical carriers and/or excipients, all of the above formulations may optionally contain one or more other therapeutically-active substances. Thus, this invention also contemplates a combination treatment regimen that relates to the administration of a composition including citicoline, or any combination of choline, cytidine, and uridine, with at least one additional therapeutic agent, or the respective pharmaceutically acceptable salts thereof.

Broad categories of the one or more optional additional therapeutic agents are contemplated. These agents include, but are not limited to, “neuroprotectives” (e.g., inhibitors of the actions of excitatory amino acids, ACEA-1021, ACPC, Aptiganel, BW-619C, CNS-1145, CNS-1505, CPC-71 and CPC-702, Dextrorphan and Dextromethorphan, Eliprodil, ES-242-1, FPL-15896, FR-115427, GP-1-4688, L-687414, L-689560, L-695902, LY-104658, LY-235959, LY-274614, LY-293558, Memantine, NNC-07-9202, NS-257, NPC 17742, “Protara”, Remacemide, Riluzole, SDZ EAA 494, Selfotel, SYM-1010, SYM-1207, YM-90K, MK-801); calcium channel blockers (e.g., AJ-394, AK-275, Calpain inhibitors, CD-349, Clentiaze, CNS-1237, CNS-2103, CPC-304 and CPC-317, Dazodipine, Diperdinine, Emopamil, Fasudil, Lacidipine, Lifarizine, Lomerizine, Magnesium, MDL:28170, NB-818, Nilvadipine, Nimodipine, NS-626 and related compounds, SM-6586, SNX-111, S-312-d, U-92032, UK-74505, US-035); agents targeted at nitric oxide; agents targeted at various other neurotransmitters (e.g., alpha₂-receptor therapeutics, CV-5197, Dopamine receptors, Enadoline, Lazabemide, Milnacipran, Nalmefene, RP-60180, SR-57746A, Synaptic uptake blockers); cytokines; hormones and related products (e.g., AN-100225 and AN-100226, Brain-derived neurotrophic factor, Calcitonin gene-related peptides, CEP075 and related compounds, Ciliary neurotrophic factor, Endothelial cell factor, Endothelin inhibitors, FR-139317 Interleukin-1 receptor antagonist (lipocortin), JTP-2942, Macrophage-regulating compounds, Motoneuronetrophic factor NBI-117, Nerve growth factor, Neural stem cells, Neutrophil inhibitory factor, NS-506, NT-3, Posatirelin, Schwann cell promoters, sCR1, Somatomedin-1); free radical scavengers (e.g., EPC-K1, MCI-186, Nicaraven, Phenazoviridin, Resorstatin, Rumbrin, Superoxide dismutase, Tirilazad mesylate, U-88999E, Yissum project P-0619, YM-737); gangliosides and related products (e.g., LIGA4, LIGA4, Monosialoganglioside (GM1), ND-37, Siagoside).

Still other classes of the one or more optional additional therapeutic agents include, but are not limited to, modulators of various specific enzymes (e.g., CEP-217, CEP-245, CEP-392, CNS-1531, Ebselen, Epalrestat, JTP-4819, K-7259, Protease nexin-1, SK-827, Tyrosine kinase modulators, Z-321); memory enhancers or “nootropics” (e.g., Aloracetam, Choline-L-alfoscerate, DN-2574, Idebenone, Oxiracetam, Piracetam, Pramiracetam, Tacrine and its analogues, Vinconate); neuroprotectives with “diverse” actions (e.g., Adomethionine sulphate tosilate, Ancrod, Apocuanzine, CPC-111, CPC-211, HSV vectors, KF-17329 and KF-19863, LY-178002, MS-153, Nicorandil, N-3393 and N-3398, SUN 4757, TJ-8007, VA-045); and imaging or contrast agents.

Therefore, methods and compositions are provided for treating a subject who is experiencing memory impairment or other forms of cognitive dysfunction, comprising administering a composition including an effective amount of citicoline, or effective amounts of choline, cytidine, and/or uridine, optionally including one or more fatty acids such as linoleic or linolenic acid that metabolize to form DAG, and at least one additional therapeutic agent, or their respective pharmaceutically acceptable salts, shortly after the onset of symptoms. More preferably, the composition is administered before symptom onset for patients at risk for developing cognitive disorders. The treatment regimen may be continued for a period of several weeks, several months, or indefinitely, depending on the condition of the patient.

The method of using the contemplated compositions includes the administration or co-administration of subsequent doses, which is preferably carried out over a period of at least about several weeks. In specific embodiment of the invention, the administration of the compositions of this invention is carried out over a period of at least about several weeks, preferably over a period of at least about several months, and most preferably over an indefinite period. Furthermore, the doses are administered one or more times daily over the treatment period. It is anticipated that subjects who may benefit the most from this therapy are those who suffer from MCI or AD.

Further, the compositions of this invention may also optionally include or be administered in conjunction with additional therapeutic agents, such as NSAIDs, gingko biloba, vitamin E, and certain B vitamins, which may be useful in treating or preventing memory impairment associated with MCI or AD.

Hence, a composition is likewise provided for the treatment of a subject experiencing memory impairment, comprising an effective amount of citicoline, or effective amounts of choline, cytidine, and/or uridine, optionally including one or more fatty acids that metabolize to form DAG, and one or more additional therapeutic agents, or their respective pharmaceutically-acceptable salts, in a pharmaceutically-acceptable carrier. In such a composition the effective amount of active ingredients may vary according to the particular needs of the patient. Typical ranges, however, may be from about 40 mg to about 4000 mg of citicoline and about 10 mg to about 500 mg of the one or more optional additional therapeutic agents.

The present invention is illustrated by the Examples that follow, it being understood, however, that the invention is not limited to the specific details of these Examples.

### 6. EXAMPLES

6.1. Animal Trial

The ability of citicoline to prevent or minimize the effects of memory impairment was demonstrated in a model of memory impairment in the rat. The impairments studied in rats are very similar to the type that occur in people with age-related memory impairment (or “minimal cognitive impairment”), as well as in more severe dementias, e.g., following stroke, brain injury or Alzheimer's Disease, in that they involve the same part of the brain, i.e., the hypothalamus. The memory impairment model in the rat was produced by separating rats weaned at 23 days old into four groups, and introducing them into either enriched or restricted environmental conditions, with or without supplementation of their diets with citicoline (500 mg/kg/day), linoleic acid, and linolenic acid for three months. Behavioral testing was conducted over a six week period during which the environmental and dietary conditions continued.

Animals from the group subjected to restricted environmental conditions that did not receive diets supplemented with citicoline exhibited a significant impairment in hippocampus-dependent types of memory. These animals were also susceptible to distraction by placing other objects in their environments. However, animals from the group subjected to restricted environmental conditions that did receive diets supplemented with citicoline, linoleic acid, and linolenic acid were protected against developing these memory impairments, and performed in a manner consistent with that exhibited by the rats raised in enriched conditions. Addition of citicoline, linoleic acid, and linolenic acid to the diets of rats raised in enriched conditions did not appear to produce any effect on ability to learn tasks, or to perform them when presented with distracting stimuli.

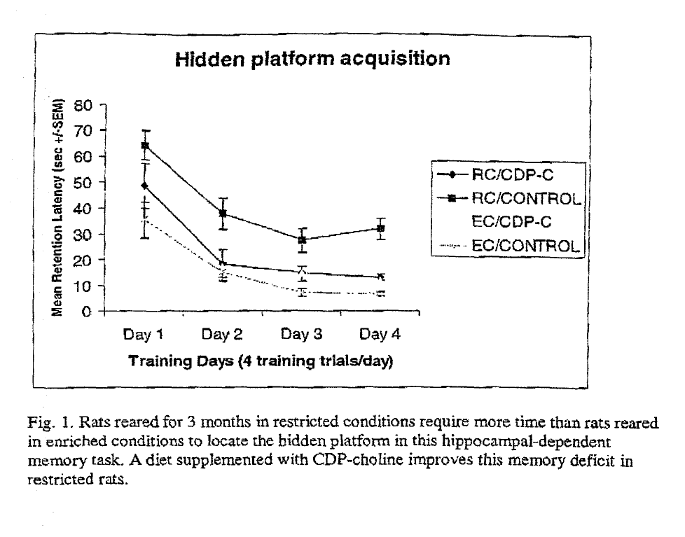

Referring to FIG. 1, this graph shows the results of behavioral testing using a hippocampal-dependent memory task. Rats reared under restricted conditions that were not provided with a diet supplemented with citicoline, linoleic acid, and linolenic acid took longer to acquire a hidden platform than rats raised in enriched conditions or rats raised in restricted conditions that received a diet supplemented with citicoline, linoleic acid, linolenic acid. This study shows that the supplementation with citicoline, linoleic acid, and linolenic acid improves the memory deficit of rats raised under restricted conditions, to the point that they are able to perform the task with efficiency similar to that of rats raised under enriched conditions.

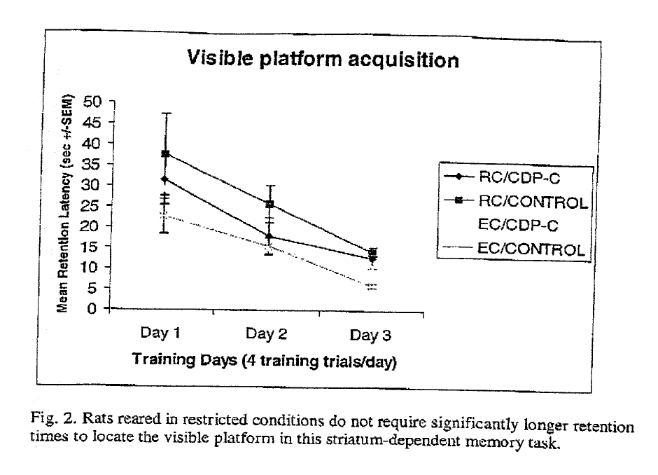

Referring to FIG. 2, when presented with a striatum-dependent memory task, rats reared in restricted conditions perform almost as well as rats raised in enriched conditions, regardless of whether they received a diet supplemented with citicoline, linoleic acid, and linolenic acid.

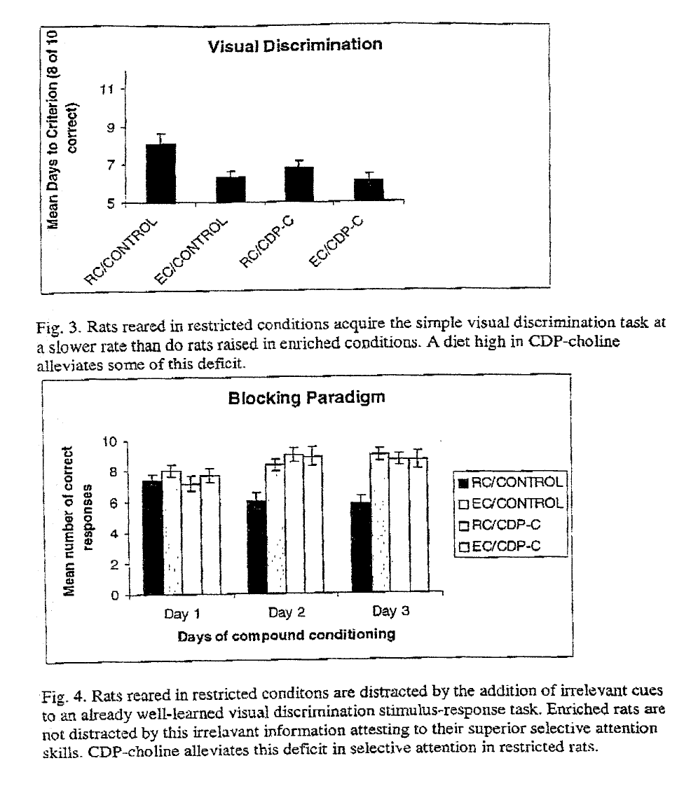

As shown in FIG. 3, rats reared in restricted conditions require a longer period of time to acquire a simple visual discrimination task than do rats raised in enriched conditions. Providing a diet supplemented with citicoline, linoleic acid, and linolenic acid alleviates some of the deficit.

FIG. 4 illustrates the effect of distraction by irrelevant cues while performing a well-learned visual discrimination stimulus-response task. Rats reared in restricted conditions were more distracted by the irrelevant information, while rats reared in enriched conditions were less distracted, evidencing their superior selective attention skills. Dietary supplementation with citicoline, linoleic acid, and linolenic acid alleviated the deficit in selective attention in rats reared in restricted conditions.

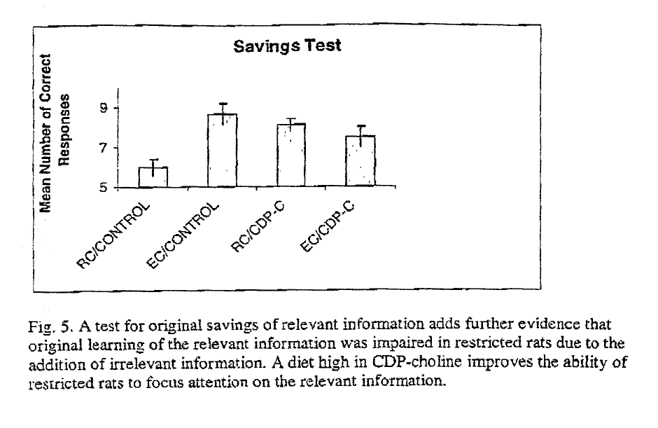

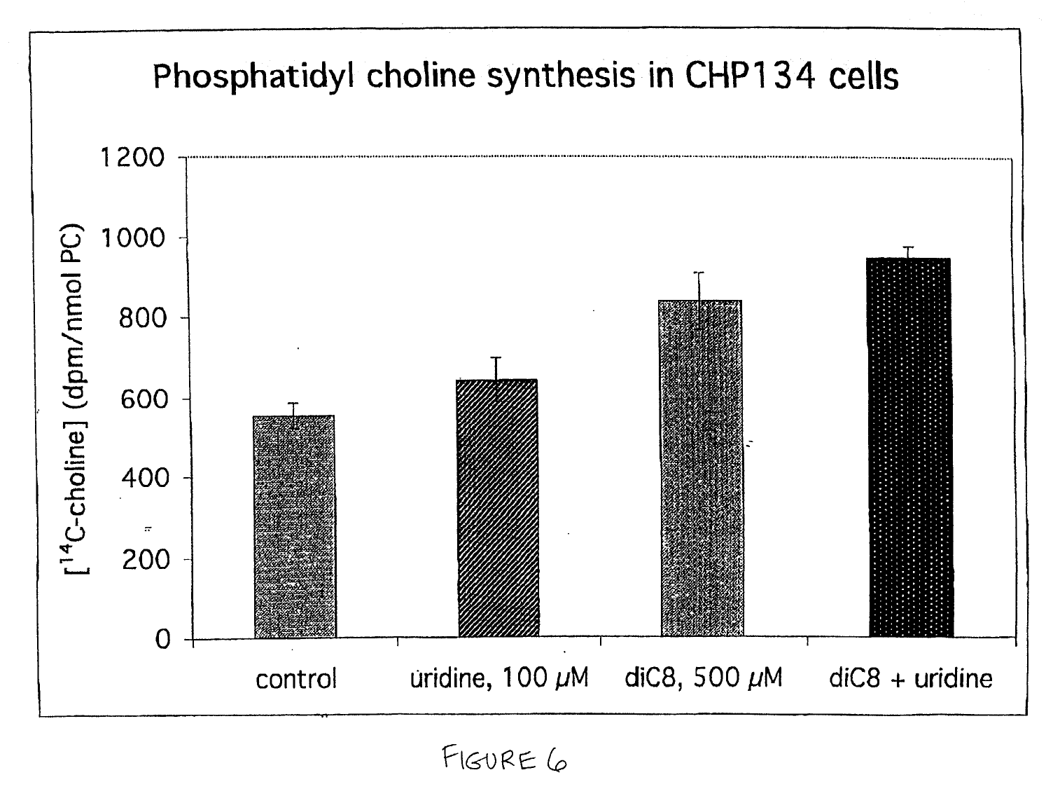

The test depicted in FIG. 5 compares the ability to learn and retain relevant information. Rats reared in restricted conditions were impaired in their ability to learn the relevant information because of the presence of additional irrelevant information. Rats reared in restricted conditions that received a diet supplemented with citicoline, linolenic acid, and linoleic acid were better able to focus attention on the relevant information.

6.2 Cell Cultures

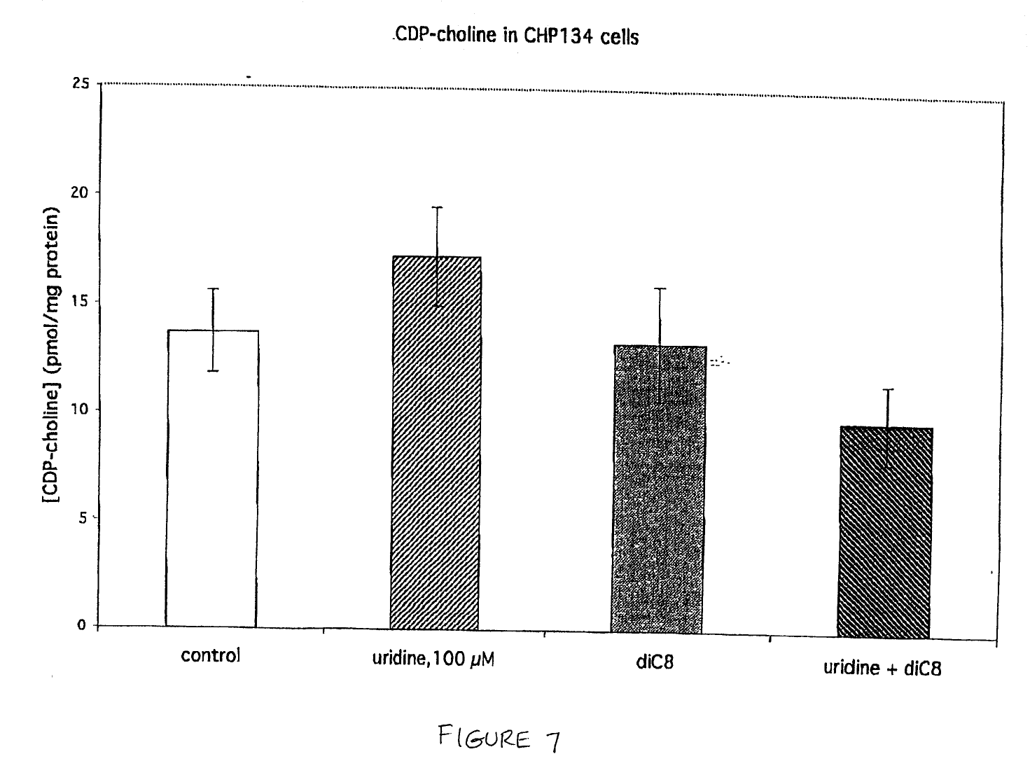

The ability of diacylglycerol (DAG, or diC8 as shown in FIGS. 6 and 7) to enhance the synthesis of phosphatidylcholine was assessed in cell cultures of the human-derived CHP 134 cell line. The cell cultures were exposed to medium containing choline, and the cell cultures were then divided into four groups according to whether no additional compounds were added to the medium (control group), 100 μM uridine was added to the medium (uridine group), 500 μM diacylglycerol was added to the medium, (diC8 group), or 100 μM uridine and 500 μM diacylglycerol were added to the medium (uridine+diC8 group).

FIG. 6 compares the amount of phosphatidylcholine formed by each of the four groups of cell lines. The results indicate that the cell culture exposed to choline, uridine, and DAG produced the highest levels of phosphatidylcholine, followed by the cell culture exposed to choline and DAG. The control cell culture showed the lowest level of phosphatidylcholine production. These results highlight the usefulness of providing a source of DAG along with choline and uridine.

FIG. 7 compares the amount of CDP-choline remaining in each of the four groups of cell lines, with lower levels indicating that more CDP-choline was converted to phosphatidylcholine. The results indicate that the cell culture exposed to choline, uridine, and DAG had the lowest levels of CDP-choline remaining, which is consistent with the results obtained in FIG. 6 that indicated that this group produced higher levels of phosphatidylcholine. The cell culture supplemented with choline and DAG exhibited lower levels of CDP-choline than did the group supplemented with choline and uridine. The control group contained CDP-choline at a level commensurate with that of the choline and DAG group. This result is not unexpected due to the role of DAG in combining with CDP-choline to form phosphatidylcholine. The absence of additional DAG in the uridine and choline-supplemented group likely resulted in increased production of CDP-choline, without enough DAG to allow all of it to be converted to phosphatidylcholine.

Other embodiments should be apparent to those of ordinary skill in view of the detailed disclosure provided herein, which embodiments would nonetheless fall within the scope and spirit of the present invention. Hence, the preceding preferred embodiments should not be construed as limiting the invention in any way.

What is claimed is:

1. A composition comprising citicoline, or a pharmaceutically-acceptable salt thereof, wherein said citicoline is metabolized to form at least one of cytidine, uridine, and choline.

2. The composition of claim 1, further comprising one or more of the compounds selected from the group consisting of linoleic acid and linolenic acid or their active metabolites.

3. The composition of claim 2, wherein the active metabolite is arachidonic acid or docsohexenoic acid.

4. A composition comprising at least one of choline or a pharmaceutically-acceptable salt thereof, cytidine or a pharmaceutically-acceptable salt thereof; and uridine or a pharmaceutically-acceptable salt thereof.

5. The composition of claim 4, further comprising a fatty acid.

6. The composition of claim 4, wherein the fatty acid is selected from the group consisting of linoleic acid and linolenic acid or their active metabolites.

7. A method for preparing a composition for use in treating or preventing memory impairment or cognitive dysfunction, comprising the steps of:
providing an effective amount of citicoline, or a pharmaceutically-acceptable salt thereof;
providing an effective amount of one or more second compounds selected from the group consisting of linoleic acid and linolenic acid; and
combining said citicoline with said one or more second compounds to form a pharmaceutically-acceptable preparation.

8. A method for preparing a composition for use in treating or preventing memory impairment or cognitive dysfunction, comprising the steps of:
providing an effective amount of choline or a pharmaceutically-acceptable salt thereof;
providing an effective amount of cytidine or a pharmaceutically-acceptable salt thereof; and
providing an effective amount of uridine or a pharmaceutically-acceptable salt thereof.

9. A method of preventing or treating cognitive disorders or memory impairment, comprising the steps of:
administering an effective amount of choline or a pharmaceutically-acceptable salt thereof;
administering an effective amount of cytidine or a pharmaceutically-acceptable salt thereof;
administering an effective amount of uridine or a pharmaceutically-acceptable salt thereof; and
continuing said administration for a period of at least about six weeks.

10. The method of claim 9, further comprising the step of administering one or more of the compounds selected from the group consisting of linoleic acid and linolenic acid.

11. A method of preventing or treating cognitive disorders or memory impairment, comprising the steps of:
administering an effective amount of choline or a pharmaceutically-acceptable salt thereof;
administering an effective amount of cytidine or a pharmaceutically-acceptable salt thereof; and
continuing said administration for a period of at least about six weeks.

12. The method of claim 11, further comprising the step of administering one or more of the compounds selected from the group consisting of linoleic acid and linolenic acid.

13. A method of preventing or treating cognitive disorders or memory impairment, comprising the steps of:
administering an effective amount of choline or a pharmaceutically-acceptable salt thereof;
administering an effective amount of uridine or a pharmaceutically-acceptable salt thereof; and
continuing said administration for a period of at least about six weeks.

14. The method of claim 13, further comprising the step of administering one or more of the compounds selected from the group consisting of linoleic acid and linolenic acid.

15. A method of preventing or treating cognitive disorders or memory impairment, comprising the steps of:
administering an effective amount of cytidine or a pharmaceutically-acceptable salt thereof;
administering an effective amount of uridine or a pharmaceutically-acceptable salt thereof; and
continuing said administration for a period of at least about six weeks.

16. The method of claim 15, further comprising the step of administering one or more of the compounds selected from the group consisting of linoleic acid and linolenic acid.

17. A method of preventing or treating cognitive disorders or memory impairment, comprising the steps of:
administering an effective amount of citicoline, or a pharmaceutically-acceptable salt thereof, combined with one or more of the compounds selected from the group consisting of linoleic acid and linolenic acid; and
continuing to administer said citicoline for a period of at least about six weeks.
